(19) United States
(12) Patent Application Publication
(10) Pub. No.: US 2012/0219579 A1
(43) Pub. Date: Aug. 30, 2012

(54) VIRUS LIKE PARTICLES COMPRISING TARGET PROTEINS FUSED TO PLANT VIRAL COAT PROTEINS

(75) Inventors: Vidadi Yusibov, Havertown, PA (US); Christine E. Farrance, Oxford, PA (US); Konstantin A. Musiychuk, Wilmington, DE (US); Vadim Mett, Newark, DE (US); Valentina Mett, Newark, DE (US)

(73) Assignee: Fraunhofer USA Inc., Newark, DE (US)

(21) Appl. No.: 13/496,568

(22) PCT Filed: Sep. 16, 2010
(86) PCT No.: PCT/US2010/049089

§ 371 Date: May 14, 2012

Related U.S. Application Data
(60) Provisional application No. 61/243,774, filed on Sep. 18, 2009.
(60) Provisional application No. 61/348,069, filed on May 25, 2010.

(51) Int. Cl. A61K 39/00; A61P 37/04; A61P 33/02; A61K 39/015; A61P 31/16; C07K 19/00; A61K 39/145

(52) U.S. Cl. 424/192.1; 530/350; 424/186.1

(57) ABSTRACT

Virus like particles comprising a fusion protein and substantially free of nucleic acid, wherein the fusion protein comprises a plant viral coat protein and a target protein, are provided. Immunogenic compositions comprising the virus like particles can be administered to subjects to induce protective immune responses in the subjects. Methods of producing the virus like particles are also provided.

### CROSS REFERENCE TO RELATED APPLICATIONS

This patent application claims priority to U.S. Provisional Application No. 61/243,774, filed Sep. 18, 2009 and U.S. Provisional Application No. 61/348,069, filed May 25, 2010, the entire contents of both of which are incorporated by reference herein.

### FIELD OF THE INVENTION

This invention relates generally to the area of recombinant vaccines. More specifically, the invention relates to virus like particles comprising target proteins fused to plant viral coat proteins for use in vaccines as well as methods for producing the virus like particles.

### BACKGROUND OF THE INVENTION

Plant viruses can be effective tools for antigen production and delivery. A variety of important protein antigens have been expressed in transgenic plants, but the level of antigenic protein produced has been relatively low. In an attempt to overcome this problem, plant viral coat proteins have been engineered to act as carrier molecules for fused antigenic peptides and used in transient expression systems. The coat proteins have the potential to self-assemble and form recombinant virus particles that display the desirable antigenic epitopes or foreign polypeptides on their surfaces. Because the recombinant viruses are replicated in cytoplasm, the desirable antigenic epitopes or foreign polypeptides might not be properly presented because of inadequate modification at the post-translational level or improper folding. Antigens produced in plants as a result of infection with recombinant tobacco mosaic viruses (TMVs) or cowpea mosaic viruses have been shown to elicit specific antibodies when injected into mice. However, in these systems, the viruses were unable to assemble when peptides greater than 25 amino acids in length were fused to their coat proteins. In a different system, fusion of polypeptides up to 47 amino acids in length to the coat protein of alfalfa mosaic virus (AIMV) did not inhibit viral assembly and the resultant viral particles containing viral nucleic acids were effective immunogens. (Yusibov, V., et al., PNAS 94: 5784-5788, 1997). More recently, a method has been developed to attach a functional fragment of protein A (133 aa) to the C-terminus of the TMV coat protein using a 15-aa peptide linker. The resultant viral constructs were able to assemble into viral particles that were capable of infecting leaf tissue and moving throughout the tissue. (Werner, S., et al., PNAS 103: 17678-17683, 2006). However, all of these systems yield infective viral particles containing viral RNA. Using such particles to elicit a protective immune response, i.e., as a vaccine, would require inoculating the subject with viral RNA as well as the intended immunogen.

There remains a need for methods and materials to present a desirable antigenic epitope or foreign polypeptide on the surface of virus like particles for immunization where the virus like particles are substantially free of viral nucleic acid.

### SUMMARY OF THE INVENTION

The disclosed subject matter of the present invention provides virus like particles as well as methods for use and preparation of the virus like particles.

According to one aspect of the present invention, a virus like particle is provided. The virus like particle comprises a fusion protein and is substantially free of nucleic acid, wherein the fusion protein comprises a plant viral coat protein and a target protein. The virus like particle may be produced in a host cell selected from the group consisting of bacterial, fungal, plant, insect, amphibian, and mammalian cells. The target protein may be fused to the N-terminus of the plant viral coat protein.

The plant viral coat protein may be derived from a coat protein of a plant virus selected from the group consisting of alfalfa mosaic virus, Johnsongrass mosaic virus, and potato virus Y.

The target protein may be derived from a polypeptide of an intracellular pathogen. The target protein may be derived from a cell surface polypeptide of Plasmodium falciparum, or a polypeptide of hemagglutinin of an influenza virus. The influenza virus may be an influenza A virus selected from the group consisting of H1N1, H3N2, H5N1, and H7N7. The influenza virus may also be an influenza B virus. The target protein may comprise an amino acid sequence selected from the group consisting of SEQ ID NOs: 4-11 and 49, and antigenic fragments thereof.

According to another aspect of the present invention, an immunogenic composition is provided. The immunogenic composition comprises the virus like particle. The immunogenic composition may further comprise an adjuvant. The adjuvant may be selected from the group consisting of aluminum salts, oil and water emulsions, and saponin-based adjuvants. In some embodiments, the plant viral coat protein is derived from a coat protein of an alfalfa mosaic virus while the target protein comprises an amino acid sequence selected from the group consisting of SEQ ID NOs: 4-11 and 49, and antigenic fragments thereof.

A method for inducing a protective immune response against an intracellular pathogen in a subject is provided. The method comprises administering to the subject an immunologically effective amount of a first composition comprising a first virus like particle, wherein the first virus like particle comprises a first fusion protein and is substantially free of nucleic acid, and wherein the first fusion protein comprises a plant viral coat protein and a first target protein derived from the first intracellular pathogen. In some embodiments, the plant viral coat protein is derived from a coat protein of an alfalfa mosaic virus while the first target protein comprises an amino acid sequence selected from the group consisting of SEQ ID NOs: 4-11 and 49, and antigenic fragments thereof. In some other embodiments, the composition further comprises an adjuvant.

The method may further comprise administering to the subject an immunologically effective amount of a second composition comprising a second virus like particle prior to the administering the first composition, wherein the second virus like particle comprises a second fusion protein and is substantially free of nucleic acid, and wherein the second fusion protein comprises the plant viral coat protein and a second target protein. The second target protein may be derived from a second intracellular pathogen that is not the first intracellular pathogen. In some embodiments, the first target intracellular pathogen may be an influenza virus, the plant viral coat protein is derived from a coat protein of an alfalfa mosaic virus, the first target protein comprises an amino acid sequence selected from the group consisting of SEQ ID NOs: 6-11, and the second target protein comprises an amino acid sequence selected from the group consisting of SEQ ID NOs: 4, 5 and 49. In particular, the first and second target proteins may comprise amino acid sequences of SEQ ID NOs: 6 and 4, respectively.

A method of producing a virus like particle in a host cell comprising a nucleotide sequence encoding a fusion protein is also provided where the virus like particle comprises the fusion protein and is substantially free of nucleic acid, and the fusion protein comprises a plant viral coat protein and a target protein. The method comprises expressing the fusion protein in the host cell under conditions that permit assembly of the virus like particle in the host cell. The method may further comprise purifying the virus like particle from the host cell.

In some embodiments, the host cell is a cell selected from the group consisting of bacterial, fungal, plant, insect, amphibian, and mammalian cells. Where the host cell is a plant cell, the method may further comprise infecting the plant cell with a recombinant bacterium capable of infecting the plant cell, wherein the recombinant bacterium comprises a nucleotide sequence encoding the fusion protein.

In some other embodiments, the target protein is derived from an intracellular pathogen.

In yet some other embodiments, the plant viral coat protein is derived from a coat protein of an alfalfa mosaic virus and the target protein comprises an amino acid sequence selected from the group consisting of SEQ ID NOs: 4-11 and 49, and antigenic fragments thereof.

### BRIEF DESCRIPTION OF THE DRAWINGS

FIG. 1 schematically illustrates fusion of a plant viral coat protein to a target protein and self-assembly of the fusion protein into a virus like particle.

FIG. 2 shows (A) an exemplary gene construct comprising a fusion protein (CP-fusion construct) and a signal sequence (PR1a); (B) cloning the CP-fusion construct into expression vector pGR-D4; and (C) cloning the CP-fusion construct into expression vector pBI121. RB and LB represent the right and left borders of the area introduced into a plant cell by a recombinant Agrobacterium.

FIG. 3 shows transmission electron micrographs of virus like particles made up of a fusion protein of an AIMV coat protein to Plasmodium falciparum Pfs25 (top panels),

Pfs28 (middle panels), or influenza virus HA3A target protein (bottom panels). The virus like particles were negatively stained (left panels) and labeled with immuno-gold tagged antibodies specific for each respective target protein (right panels). Bar=100 nm or 200 nm as labeled.

FIGS. 4A and 4B show immunoblots of purified fusion proteins of AIMV coat protein fused with (A) Pfs25 or (B) HA3A. The right half of each blot was incubated with an antibody against AIMV. The left half of each blot was incubated with a monoclonal antibody to (A) Pfs25 or (B) HA3A. FIG. 4C is an immunoblot of crude extract of plant proteins containing the AIMV coat protein fused with the full length hemagglutinin HAA target protein extracted under different conditions to assess solubility. The blot was reacted with an anti-HAA monoclonal antibody. TSP=total soluble protein; TSP-T=triton-extracted soluble protein; TP=total protein.

FIG. 5A shows anti-A/Anhui/1/05 IgG responses in sera samples from mice 28 or 56 days after immunization with VLPs containing HA3A-CPF (“CPF” designates “coat protein fusion”) with or without an adjuvant, QUIL A™ or ALHYDROGEL™, or HAA1 with QUIL A™. FIG. 5B shows serum hemagglutination inhibition (HI) antibody responses in sera samples from the same mice as in FIG. 5A.

FIG. 6 shows IgG titers in sera collected from mice 56 days after immunization with VLPs containing Pfs25-CPF or Pfs28-CPF, with or without an adjuvant, QUIL A™ or ALHYDROGEL™.

FIG. 7 shows anti-A/Indonesia/5/05 IgG isotype titers in sera samples collected from mice 35 days after immunization with HAI3-CPF VLPs or HAI1, with or without ALHYDROGEL™, or CP only.

FIG. 8 shows serum hemagglutination inhibition (HI) antibody responses in sera samples from mice after immunization with HAI3-CPF VLPs or HAI1, with or without ALHYDROGEL™, or CP only.

FIGS. 9A and 9B show serum hemagglutination inhibition (HI) antibody responses against A/Anhui/1/05 in sera samples from mice after a primary vaccination with Pfs25-CPF VLPs or PBS, with or without ALHYDROGEL™, and a secondary vaccination with PBS or HA3A-CPF VLPs, with or without ALHYDROGEL™.

### DETAILED DESCRIPTION OF THE INVENTION

The present invention generally relate to virus like particles (VLPs) comprising a fusion protein that is substantially free of nucleic acid, and to methods of using and preparing the VLPs.

The term “virus like particle” or “VLP” used herein refers to a nonreplicating viral shell derived from a virus, for example, a plant virus as discussed below. VLPs are generally composed of at least one viral protein (e.g., capsid, coat, shell, surface or envelope protein), which can form spontaneously upon expression of the viral protein in a variety of suitable host cells, for example, bacterial, fungal, plant, yeast, insect, amphibian, and mammalian cells. The presence of the VLPs may be detected using conventional techniques known in the art (e.g., electron microscopy, dynamic light scattering, and size-exclusion chromatography). VLPs may also be isolated using conventional techniques in the art (e.g., density gradient centrifugation, size-exclusion chromatography, and affinity chromatography).

The terms “protein” and “polypeptide” are used herein interchangeably, and refer to a polymer of amino acid residues with no limitation with respect to the minimum length of the polymer. The definition includes both full-length proteins and fragments thereof, as well as modifications thereof (e.g., glycosylation, phosphorylation, deletions, additions and substitutions).

The term “derived from” used herein refers to the origin or source, and may include naturally occurring, recombinant, unpurified or purified molecules.

The term “fragment” of a protein as used herein refers to a polypeptide having an amino acid sequence that is the same as a part, but not all, of the amino acid sequence of the protein.

The term “variant” of a protein as used herein refers to a polypeptide having an amino acid sequence that is the same as the amino acid sequence of the protein except having at least one amino acid modified, for example, deleted, inserted, or replaced. The variant may have an amino acid sequence at least about 80%, 90%, 95%, or 99%, preferably at least about 90%, more preferably at least about 95%, identical to the amino acid sequence of the protein.

The term “antigen” as used herein refers to a molecule containing one or more epitopes (linear and/or conformational) that are capable of stimulating the immune system of a subject to make a humoral and/or cellular antigen-specific response. A humoral immune response refers to an immune response mediated by antibodies produced by B lymphocytes, or B cells, while a cellular immune response refers to an immune response mediated by T lymphocytes, or T cells, and/or other white blood cells. In general, a B-cell epitope contains at least about 5 amino acids but can be 3-4 amino acids while a T-cell epitope includes at least about 7-9 amino acids and a helper T-cell epitope includes at least 12-20 amino acids. An antigen may be derived from a protein (e.g., a surface protein) of an intracellular pathogenic organism or pathogen.

The term “immunogenic composition” as used herein refers to a composition that comprises an antigenic molecule and is capable of eliciting a humoral and/or cellular antigen-specific response in a subject upon administration of the composition to the subject.

The term “subject” as used herein refers to a mammal, preferably a human.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate to perform the disclosed methods.

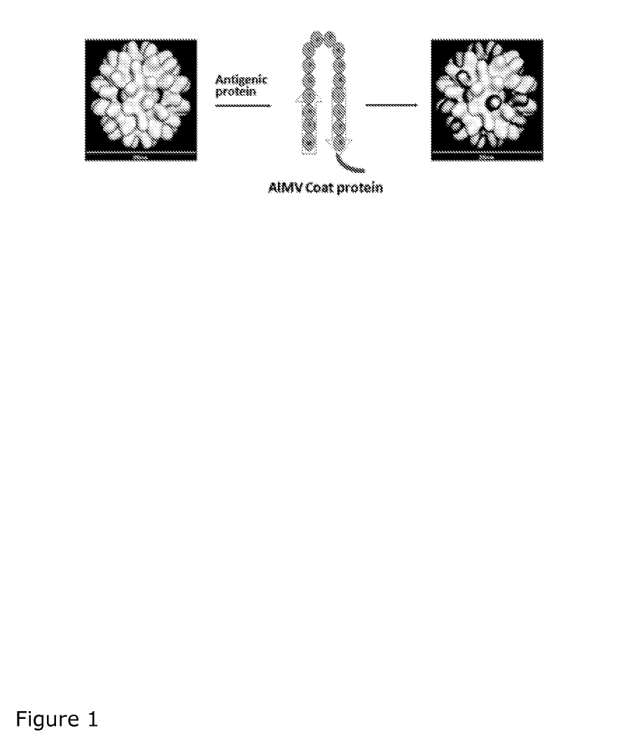

According to one aspect of the present invention, a virus like particle (VLP) is provided. The VLP comprises a fusion protein and is substantially free of nucleic acid, wherein the fusion protein comprises a plant viral coat protein and a target protein. FIG. 1 demonstrates schematically that fusion of a target protein with a plant viral coat protein results in assembly of a VLP presenting the target protein on the surface of the VLP.

The plant viral coat protein may be derived from a coat protein of any virus that is capable of self-assembling into particles substantially free of nucleic acid. The plant viral coat protein may be a full length coat protein, or a functional fragment or variant thereof. Examples of suitable plant viral coat proteins include proteins derived from coat proteins of alfalfa mosaic virus (AIMV-CP, CP or CPF), potato virus Y (Stram, et al., Virus Research 28: 29-35, 2002) and Johnsongrass mosaic virus (Jagadish, et al., J. Gen. Virol. 74: 893-896, 1993).

The amino acid sequence of a coat protein may be genetically modified to improve particle assembly or immunogenicity. The amino acids of the coat protein may be deleted, inserted, or replaced, provided that the resulting variant of the coat protein retains the ability to assemble into a particle in the absence of nucleic acid when fused to a target protein. A fragment or variant of a coat protein is functional where the fragment or variant is capable of self-assembling into a particle that is substantially free of nucleic acid. For example, the protein represented by SEQ ID NO:1 lacks the first methionine (from the start codon) of the native AIMV-CP sequence, and SEQ ID NO:2 is a truncated fragment of the AIMV-CP sequence that is missing the first 25 amino acids of the native protein sequence.

Target proteins may be derived from any desirable antigen or polypeptide of any source. They may have at least about 6, 10, 50, 100, 200, 300, or 500 amino acids. Preferred target proteins are derived from surface proteins from intracellular pathogenic organisms, e.g., bacterial, viral, fungal, and parasitic organisms that are suitable for use in vaccines. Examples of viral organisms include Plasmodium falciparum and influenza viruses. Influenza viruses include various strains of influenza A viruses (e.g., H1N1, H3N2, H5N1, and H7N7) and influenza B viruses. A target protein derived from a surface protein may be the full length surface protein or a fragment or variant thereof. For example, the target protein may be derived from a cell surface polypeptide of Plasmodium falciparum, or a polypeptide of hemagglutinin of an influenza virus.

For use in a vaccine, preferred fragments or variants are those that are antigenic, i.e., capable of inducing a protective immune response in a subject. The fragments or variants have at least about 6, 7, 8, 9, 10, 11, 12, 13, 14 or 15 amino acids in length. Any protein, protein fragment, or protein variant may be used as a target protein, as long as particles substantially free of nucleic acid are self-assembled when it is fused to a plant viral coat protein. Some examples of target proteins are described by SEQ ID NOs: 4-19 and 23-49 in the Sequence Listing.

The target fusion protein may further comprise a signal sequence to target the fusion protein to a particular site in a host cell (e.g., ER, chloroplast) or to direct extracellular secretion of the fusion protein. The absence of a signal peptide results in translation of the proteins in the cytoplasm. Any signal sequence appropriate for the host cell may be utilized, or the signal sequence may be omitted. Signal sequences have been found to be conserved across phyla and kingdoms, and, in general, almost any signal sequence may be used. Bennett and Scheller, PNAS 90: 2559-2563, 1993; Luirink and Sinning, Biochim. Biophys. Acta 1694: 17-35, 2005; Doudna and Batey, Ann. Rev. Biochem. 73: 539-557, 2004; Stern, et al., Trends in Cell and Mol. Biol. 2: 1-17, 2007. In one embodiment for expression in a plant tissue, a preferred signal sequence is the plant pathogenesis-related protein 1 (PR-1) of Nicotiana tabacum (SEQ ID NO:3).

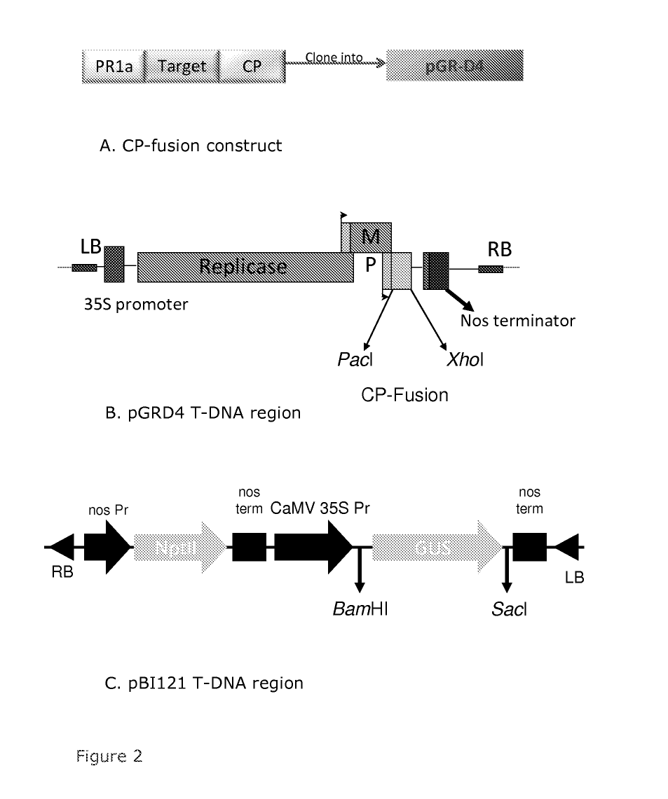

The fusion proteins can be prepared using standard methods of cloning and molecular biology, e.g., as described in Example 1. An exemplary construct containing coding sequences for a plant viral coat protein and a target protein may be prepared and subcloned into an expression vector (FIG. 2). The target protein may be fused to the plant viral coat protein directly or indirectly (e.g., via a peptide linker). For example, the target protein may be fused to the N-terminus of the plant viral coat protein by placing the coding sequence for the target protein at the 5′ end of the code sequence of the plant viral protein (FIG. 2). The expression construct may optionally include a signal sequence to direct the localization or secretion of the fusion protein. The expression vector may be introduced into a suitable expression system for expression of the fusion protein in a host cell using convention techniques known in the art (e.g., transfection, transduction, infection and electroporation). Any expression system appropriate for a selected host cell may be used, including plasmid and viral vectors. Suitable host cells may include bacterial, mammalian, plant, fungal, amphibian and insect cells. The use of plasmid and viral vectors for expression of the fusion proteins in plant cells is described in Examples 1-3. Transgenic organisms may also be created to produce the fusion proteins. Appropriate expression vectors and methods for producing recombinant proteins in these host systems and for isolating and purifying the recombinant proteins are known in the art and are described, for example, in the “Practical Approach” series published by Oxford University Press, which includes Protein Expression, a Practical Approach, S. J. Higgins and B. D. Hames, Eds., 1999; Molecular Plant Biology Vols. 1 and 2, P. M. Gilmartin and C. Bowler, Eds., 2002; Protein Purification Techniques, A Practical Approach, S. Roe, Ed., 2001; and the “Methods in Molecular Biology/Medicine” series published by Humana Press, which includes Transgenic Plants: Methods and Protocols, Leandro Peña, Ed., 2004; Baculovirus and Insect Cell Expression Protocols, D. W. Murhammer, Ed., 2007; and Adenovirus Methods and Protocols, Vols. 1 and 2, W. S. M. Wold and A. E. Tollefson, Eds., 2007.

The invention also encompasses an isolated polynucleotide that encodes a fusion protein comprising a plant viral coat protein and a target protein. The polynucleotides may be obtained by any appropriate means known to the art. For example, the nucleotide sequence of a target protein or a fragment or variant thereof, or the nucleotide sequence of a coat protein or a fragment or variant thereof may already be known or may be determined by screening a library and sequencing a potential target or coat protein. Alternatively, the amino acid sequence of a particular fusion protein may be reverse-translated to obtain appropriate nucleotide sequences and corresponding nucleotides may be prepared synthetically.

The VLPs of the present invention are especially advantageous for antigen presentation. The VLPs are highly immunogenic. Further, the VLPs are substantially free of nucleic acid. For example, the particles may appear empty as being visualized by, for example, TEM and negative staining. The particles may contain less than about 10%, 5%, 1%, or 0.1%, preferably less than about 5%, more preferably less than about 1%, nucleic acid by weight. Accordingly, they provide safe and effective vaccines against viral, bacterial, and eukaryotic pathogens.

An immunogenic composition comprising the VLPs is provided. The immunogenic composition is capable of inducing an immune response (i.e., a humoral and/or cellular response) against the target protein in a subject upon administration of the composition to the subject. In some embodiments, the target protein is derived from an intracellular pathogen, and the immunogenic composition is capable of eliciting a protective immune response to the pathogen in a subject upon administration of the composition to the subject.

The immunogenic composition can be formulated as pharmaceutical compositions comprising an effective amount of the VLPs, a pharmaceutically acceptable carrier, diluent, and/or excipient. An “effective amount” refers to an amount of the VLPs required to exhibit a detectable immunopotentiating effect. The effective amount will depend on route of administration, the nature of the formulation, and the subject's condition. A suitable range of “effective amount” may be from about 0.001 to about 100 μg virus like particles (or fusion protein particles) per kg body weight of the subject. Suitable carriers, diluents, and excipients are known in the art and include, but are not limited to, saline, buffered saline, mannitol, L-histidine, polysorbate 80, dextrose, water, glycerol, ethanol, and combinations thereof. The composition may optionally contain an adjuvant. Appropriate adjuvants include aluminum salts (e.g., Alhydrogel™), oil and water emulsions, and saponin-based adjuvants (Quil A™)

The immunogenic composition may be formulated for oral, anal, transdermal, nasal, mucosal, or parenteral administration. Parenteral administration includes subcutaneous, intramuscular, intraperitoneal, and intravenous injection.

A method for inducing a protective immune response against a first intracellular pathogen in a subject is also provided. The method comprises administering to the subject a first immunogenic composition comprising a first virus like particle, wherein the first virus like particle comprises a first fusion protein and is substantially free of nucleic acid, and wherein the first fusion protein comprises a plant viral coat protein and a first target protein derived from the first intracellular pathogen. The composition may further comprise an adjuvant.

The method may further comprise, prior to the administering the first immunogenic composition, administering to the subject a second immunogenic composition comprising a second virus like particle, wherein the second virus like particle comprises a second fusion protein and is substantially free of nucleic acid, and wherein the second fusion protein comprises the plant viral coat protein and a second target protein. The second target protein may be derived from a second intracellular pathogen that is not the first intracellular pathogen. In some embodiments, the target intracellular pathogen is a strain of an influenza virus while the first target protein is derived from a different intracellular pathogen (e.g., a malaria parasite, Plasmodium falciparum) or a different strain of the influenza virus. For example, the target pathogen is an influenza virus, the plant viral coat protein is derived from a coat protein of an alfalfa mosaic virus, the first target protein comprises an amino acid sequence of SEQ ID NO: 6, 7, 8, 9, 10 or 11, and the second target protein comprises an amino acid sequence of SEQ ID NO: 4, 5 or 49.

A method of producing the VLP in a host cell is further provided. The method comprises expressing a fusion protein, which comprises a plant viral coat protein and a target protein, in a host cell comprising a nucleotide sequence encoding the fusion protein under conditions that permit assembly of the virus like particle (VLP) in the host cell. For example, the VLP may be assembled in a plant tissue about 3-7 days after the plant is infiltrated with an expression vector bearing the corresponding fusion construct. The VLP comprises the fusion protein and is substantially free of nucleic acid. The method may further comprise purifying the VLP from the host cell. Suitable host cells include bacterial, fungal, plant, insect, amphibian, and mammalian cells. The nucleotide sequence encoding the fusion protein may or may not be integrated into the host cell genome. Where the host cell is a plant cell, the method may further comprise infecting the plant cell with a recombinant bacterium capable of infecting the plant cell (e.g., A. tumefaciens strain GV3101 and other infectious A. tumefaciens strains, and infectious A. rhizogenes strains), wherein the recombinant bacterium comprises a nucleotide sequence encoding the fusion protein.

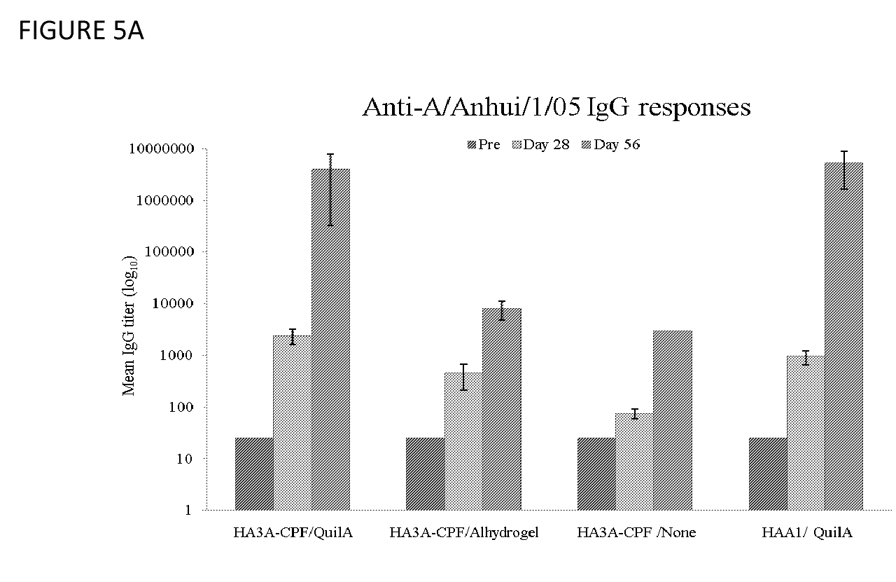

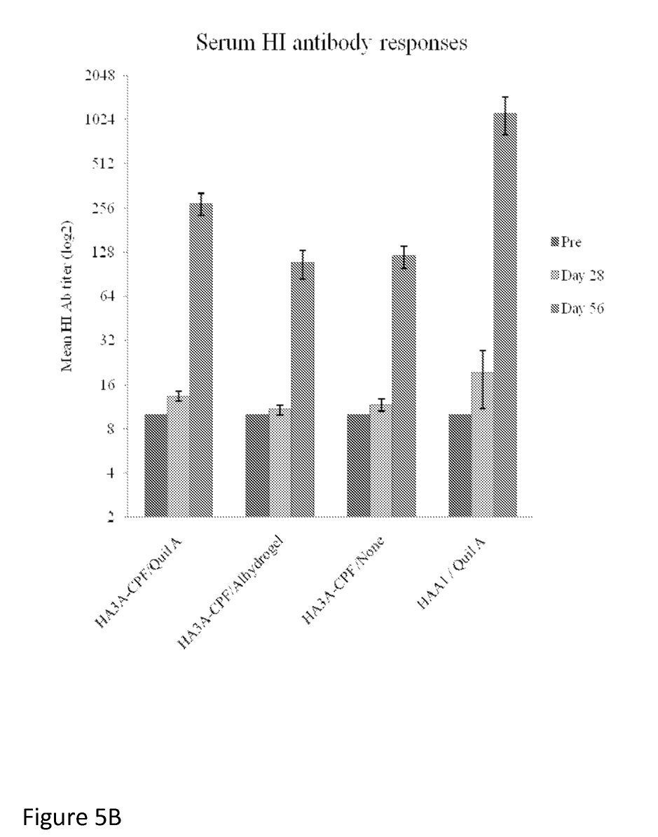

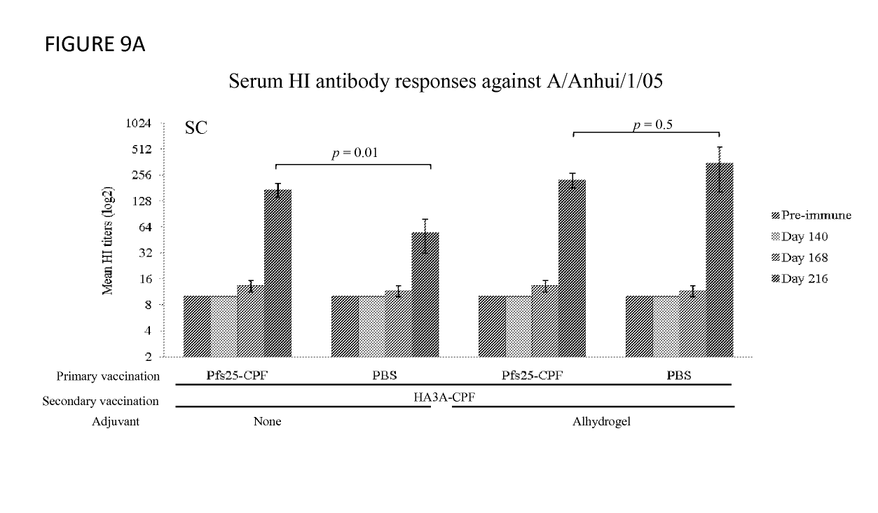

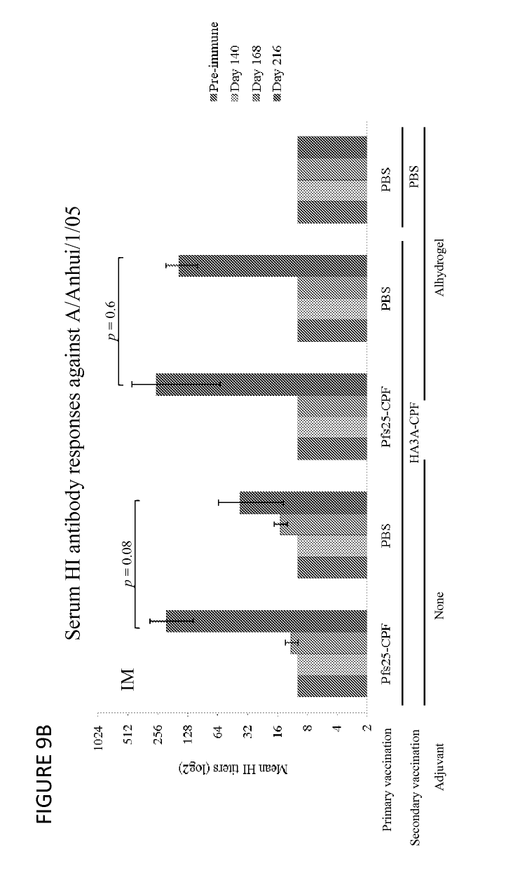

Preparation and testing of exemplary fusion proteins comprising target proteins derived from a malaria parasite, Plasmodium falciparum, an influenza virus A/Anhui/1/2005(H5N1), and an influenza virus A/Indonesia/5/2005(H5N1) are described in examples below. When these target proteins were fused with the AIMV coat protein, the resultant fusion proteins assembled into apparently empty VLPs. These particles bound antibodies specific to AIMV and specific to the target proteins. Sera from mice immunized with the VLPs containing the malaria target proteins produced high titers of parasite-binding antibodies and were capable of preventing transmission of the parasite in mosquitoes (Tables 2 and 3). Mice immunized with VLPs containing the influenza proteins produced protective levels of antibody titers as described in Examples 4 and 8, and shown in FIGS. 5, 7 and 8. Pre-existing immunity against a plant viral coat protein did not interfere with the protective levels of antibody titers in mice immunized with VLPs containing the same plant viral coat protein as described in Example 9 and shown in FIG. 9.

### EXAMPLE 1

### Construction of a Heterologous Vector for the Expression of AIMV-CP Fusion Proteins

Target genes included cell surface proteins specific to different sexual stages of the malaria parasite, P. falciparum, (Pfs25 (SEQ ID NO:4), Pfs28 (SEQ ID NO:5), and Pfs230 (SEQ ID NO: 49)), the globular domain of hemagglutinin (HA) from the Anhui strain of influenza virus (HA3A (SEQ ID NO:6)), the globular domain of HA from the California strains of influenza virus (HA3CO4 (SEQ ID NO:7) and HA3C06 (SEQ ID NO:8)), the globular domain of HA from the Indonesia strains of influenza virus (HA3I (SEQ ID NO:11)), full length HA from the Anhui strain (HAA or HAA1 (SEQ ID NO:9)), and full length HA from the Indonesia strain (HAI or HAI1 (SEQ ID NO:10)). Each target gene was cloned, using standard methods of molecular biology, as an N-terminal fusion to the AIMV coat protein (AIMV-CP, CP or CPF) or the optimized AIMV coat protein (CPO), which is encoded by a coding sequence optimized for expression in plants, in a shuttle vector with Pad and BsmBI restriction enzymes. FIG. 2 shows an exemplary structure of the fusion protein constructs. Sequences were verified by automated sequencing.

Each target-CP sequence was then subcloned into an expression vector, which optionally encoded the signal peptide of the plant pathogenesis-related protein 1 (PR-1) (SEQ ID NO:3). Specifically the sequence was subcloned into the BamHI-SacI sites of pBI121 (Clontech), or into the PacI-XhoI sites of pGRD4 (described in Shoji, et al., Vaccine 27: 108701092, 2009). The cloning strategy is shown in FIG. 2.

### EXAMPLE 2

### Infiltration of Plants with Expression Vectors Bearing Fusion Constructs

The expression vectors produced in Example 1 were then introduced into Agrobacterium tumefaciens strain GV3101 and the resulting bacteria were grown overnight in minimal medium. The optical density of the cultures was determined and the protein expression strain was mixed with an Agrobacterium strain expressing the suppressor of silencing protein, p19, at a 4:1 ratio to a final O.D. of 0.5. The Agrobacterium solution was introduced by hand infiltration into the aerial parts of 6 week old, soil-grown, Nicotiana benthamiana plants as described previously (Green, et al., Biotechnol. J. 4: 1-8, 2009).

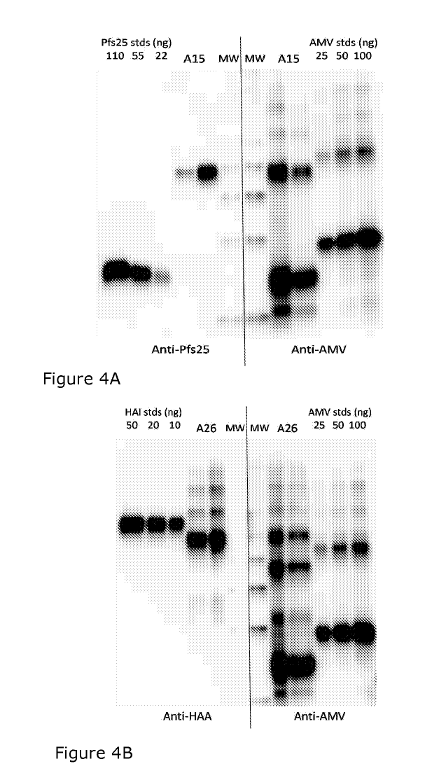

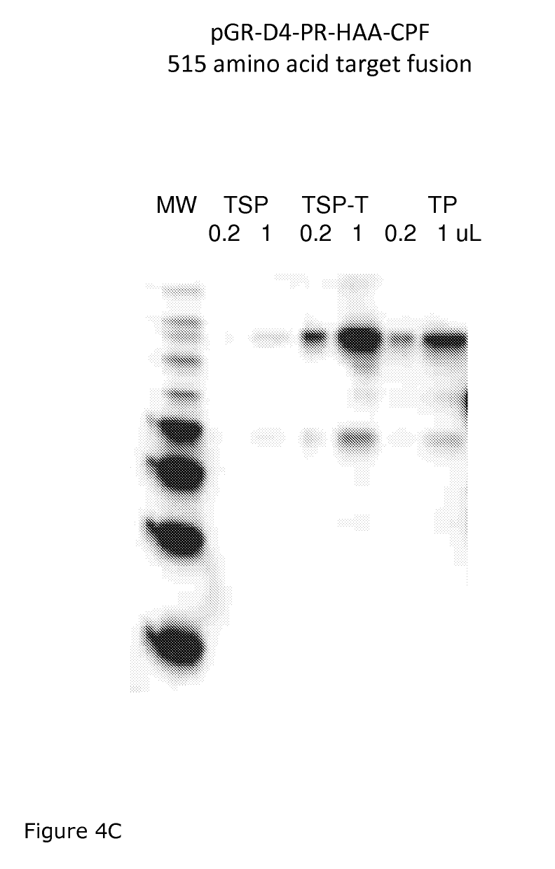

Plant tissue samples were taken from 3-7 days post-infiltration for determination of the levels of expression and solubility of the fusion protein. Samples were weighed and extracted in three volumes of extraction buffer (100 mM Na₂HPO₄, pH 7.1; 2.5 mM EDTA, pH 8.0) for total soluble protein, extraction buffer plus 0.5% Triton-X 100 for total soluble protein-Triton, or gel loading buffer for total protein. Proteins were resolved on 10% SDS-PAGE gels and transferred to PVDF membranes. Levels of fusion protein were assessed by immunoblot analysis with anti-AIMV rabbit polyclonal antibody compared with an AIMV standard or with a target specific antibody and appropriate standard. A representative immunoblot is shown in FIG. 4C. Peak days of expression and solubility profiles were determined. Constructs of interest were vacuum infiltrated on a larger scale into hydroponically grown N. benthamiana for expression of the fusion proteins.

### EXAMPLE 3

### Isolation and Purification of Virus Like Particles

On the day of maximum expression, leaves were harvested and homogenized in a blender in three volumes of phosphate-based extraction buffer with 0.5% Triton X-100. The homogenate was stirred for 30 minutes at 4° C., then centrifuged for 30 minutes at 5,000×g. The supernatant was filtered through miracloth and centrifuged at 15,000×g for 1 to 1.5 hours. The supernatant was precipitated with PEG and then again centrifuged for 30 min at 15,000×g. The pellet was resuspended in a phosphate based buffer and frozen at −20° C. After thawing, the suspension was centrifuged 30 minutes at 30,000×g. Aliquots of supernatant were analyzed by SDS-PAGE to estimate protein concentration. Supernatant was centrifuged for 2 h at 60,000×g in a Ti70 rotor. The pellet was resuspended in phosphate-based buffer. The resulting supernatant was analyzed by SDS-PAGE and Coomassie blue staining. Protein concentration was determined colorimetrically by comparison with BSA standards.

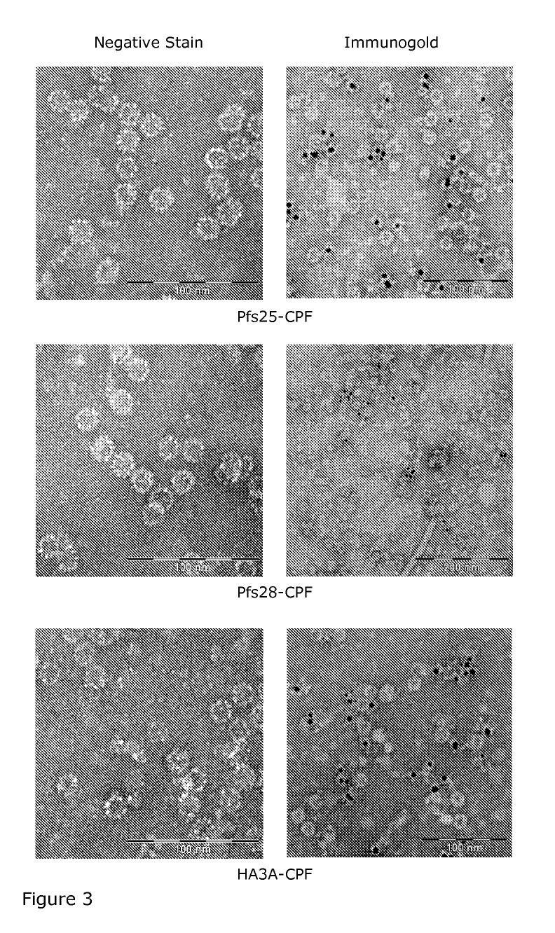

Purified particles were visualized by TEM and negative staining (FIG. 3). The particles appear to be empty and substantially free of nucleic acid. Immuno-gold labeling with target-specific antibodies confirmed the presence of the target protein on the surface of the particles. Purified particles were stored in a buffer solution of 10 mM Na₂HPO₄, pH 7.1; 1 mM EDTA, pH 8.0, at −80° C.

The purified proteins were subjected to immunoblotting as described in Example 3. On immunoblots having the purified particles, the fusion protein was recognized by both the target specific antibody and by the AIMV specific antibody, which binds to the coat protein. (FIGS. 4A and 4B).

Expression results of fusion proteins produced in leaves are summarized in Table 1. Expression and purification amounts were sufficiently high to allow testing for immunogenicity in mice.

TABLE 1
Protein expression from fusion constructs infiltrated into plant leaves

| | | | Target | |
|---|---|---|---|---|
| | | | protein | Total |
| | Target | | size in | Protein |
| | SEQ ID | | amino | Expression |
| Fusion construct | No. | Target protein | acids | (mg/kg) |
| pGR-D4-PR-Pfs25-CPF | 4 | P. falciparum sexual stage | 171 | 130 |
| | | protein, Pfs25 | | |
| pBI-PR-Pfs25-CPF | 4 | P. falciparum sexual stage | 171 | 174 |
| | | protein, Pfs25 | | |
| pGR-D4-Pfs28-CPF | 5 | P. falciparum sexual stage | 175 | 101 |
| | | protein, Pfs28 | | |
| pGR-D4-PR-Pfs28-CPF | 5 | P. falciparum sexual stage | 175 | 79 |
| | | protein, Pfs28 | | |
| pBI-PR-Pfs28-CPF | 5 | P. falciparum sexual stage | 175 | 100 |
| | | protein, Pfs28 | | |
| pBI-PR-HA3A-CPF | 6 | Hemagglutinin globular | 233 | 75 |
| | | domain | | |
| | | A/Anhui/1/2005(H5N1) | | |
| pGR-D4-PR-HA3A-CPF | 6 | Hemagglutinin globular | 233 | 60 |
| | | domain | | |
| | | A/Anhui/1/2005(H5N1) | | |
| pGR-D4-PR-HA3C04-CPF | 7 | Hemagglutinin globular | 234 | 14 |
| | | domain | | |
| | | A/California/04/2009(H1N1) | | |
| pGR-D4-PR-HA3C06-CPF | 8 | Hemagglutinin globular | 234 | 17 |
| | | domain, | | |
| | | A/California/06/2009(H1N1) | | |
| pGR-D4-PR-HAA-CPF | 9 | Hemagglutinin | 515 | 4 |
| | | A/Anhui/1/2005(H5N1) | | |
| pGR-D4-PR-HA3I-CPF | 11 | Hemagglutinin globular | 233 | 4 |
| | | domain | | |
| | | A/Indonesia/5/2005(H5N1) | | |
| PGR-D4-PR-HA3I-CPO | 11 | Hemagglutinin, | 233 | 24 |
| | | A/Indonesia/5/05(H5N1) | | |
| pGR-D4-PR-230AM-CPO | 49 | P. falciparum sexual stage | 444 | 23 |
| | | protein, Pfs230 | | |

### EXAMPLE 4

### Immunization of Mice with Virus Like Particles

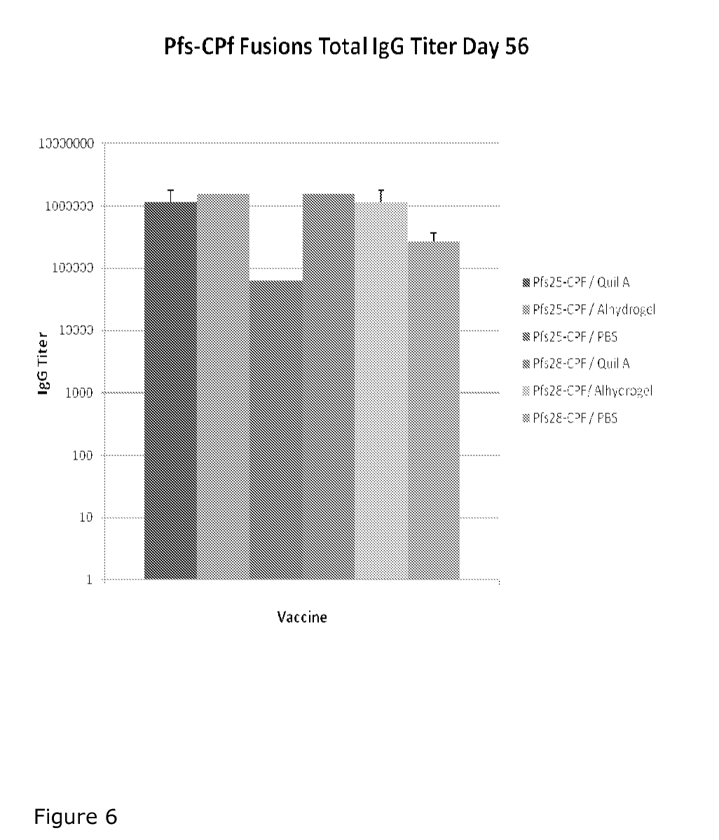

Groups of six, 6-8 weeks old Balb/c mice were immunized subcutaneously on study days 0 and 28 with 10 μg of virus like particles containing Pfs25-CPF or Pfs28-CPF or 1 μg of virus like particles containing HA3A-CPF. The vaccine was prepared just before using. Aliquots of purified virus like particles (or fusion protein particles) were thawed in buffer and injected with or without an adjuvant. The vaccine was injected in PBS alone or in the presence of either QUIL A™ (saponin based adjuvant, Brenntag Biosector, Frederikssund Denmark) or ALHYDROGEL™ (aluminum hydroxide based adjuvant, Brenntag Biosector, Frederikssund Denmark) as an adjuvant. The full length soluble HA from the Anhui strain of influenza virus (HAA1) was also injected with QUIL A™ as a positive control. Serum samples were collected on study day 56 and analyzed for antigen-specific IgG responses by ELISA. In the assay, plates were coated with 1 μg/ml of the following proteins: recombinant Pfs25 or Pfs28 produced in Pichia pastoris, or coated with inactivated A/Anhui/1/2005 reassortant virus obtained from the CDC at a 1:128 dilution. Serum samples were plated in 4-fold dilutions down the plate. End point titers of IgG were determined as the dilution at which the corresponding OD values are greater than four times the blank value. Results demonstrated that high antibody titers (>10,000) were elicited by Pfs25-CPF and Pfs28-CPF on Day 56 with or without an adjuvant (FIG. 6). HA3A-CPF also elicited antigen-specific antibody titers in the presence or absence of an adjuvant (FIG. 5A).

To test the functionality of the antibodies elicited by the HA3A-CPF vaccine, hemagglutination inhibition (HI) assays were performed as described in Yoko et al. (Vaccine 27: 3467-3470, 2009). Results showed that all mice immunized with HA3A-CPF in PBS or QUIL A™ had an HI titer of >1:40, a level considered protective in humans (FIG. 5B). Four out of five animals immunized with HA3A-CPF vaccine adsorbed to ALHYDROGEL™ had an HI titer >1:40. Based on these results, HA3A-CPF appears to be a viable candidate for a pandemic influenza vaccine that can be delivered in the absence of an adjuvant.

Efficacy of the antibodies elicited by the Pfs25-CPF and Pfs28-CPF vaccines were evaluated using a different group of assays. IgG titers as determined by ELISA in sera collected on day 56 are shown in FIG. 6. Additionally, these serum samples were sent to Radboud University, Nijmegen, The Netherlands, to assess the presence of gametocyte-binding antibodies. Initial testing of sera was done using an immunofluorescence assay (IFA) on fixed gametocytes where a titer of parasite binding antibodies in each serum sample was determined (Table 2). Positive sera from the IFA assay was then tested in a suspension IFA assay (SIFA) using live gametocytes at a serum dilution of 1:50 and 1:250. SIFA results are shown in Table 2.

TABLE 2
Efficacy of Malaria CP-Fusion Vaccines

| Vaccine | | Dose | IFA (Ab | SIFA (Ab |
|---|---|---|---|---|
| candidate | Adjuvant | (μg) | Titer) | Titer) |
| Pfs25-CPF | Quil A | 10 | Positive | Very |
| | | | (6400) | Positive |
| | | | | (250) |
| Pfs25-CPF | Alhydrogel | 10 | Positive | Very |
| | | | (6400) | Positive |
| | | | | (250) |
| Pfs25-CPF | PBS | 10 | Positive | Very |
| | | | (800) | Positive |
| | | | | (250) |

The sera were then tested for the presence of transmission blocking antibodies in a standard membrane feeding assay (SMFA). The SMFA assay is the gold standard for measurement of transmission-blocking activity in test sera. The bioassay mimics the natural infection situation, and is therefore considered to be a valuable assay to measure transmission between man and mosquito. This assay enables transmission blocking vaccine (TBV) development to proceed with a confidence that is not available to most other malaria vaccines. Briefly, laboratory-reared Anopheles stephensi mosquitoes are allowed to take a blood meal from membrane-covered devices that contain serum from the above-immunized mice combined with complement and red blood cell suspensions infected with P. falciparum gametocytes. After one week, the number of infected mosquitoes (% InfMosq), as well as the number of developed oocysts per mosquito is determined. Transmission reducing activity is determined by comparing oocyst numbers in mosquitoes that are fed with test versus control (preimmune) serum. These results are shown in Table 3.

TABLE 3
Standard Membrane Feeding Assay

| Vaccine | | IFA | | SMFA | | | | |
|---|---|---|---|---|---|---|---|---|
| candidate | Adjuvant | Ab Titer | SIFA | No. | Total | % | AMO/ | |
| — | — | — | — | mosqs | Oocysts | InfMosq | feeder | Pvalue |
| Pfs25-CPF | Quil A | Positive | Very | 20 | 0 | 0 | 0 | P < 0. [symbol] |
| | | (6400) | Positive | | | | | 01 |
| | | | (250) | | | | | |
| Pfs25-CPF | Alhydrogel | Positive | Very | 20 | 0 | 0 | 0 | P < 0. [symbol] |
| | | (6400) | Positive | | | | | 01 |
| | | | (250) | | | | | |
| Pfs25-CPF | PBS | Positive | Very | 20 | 0 | 0 | 0 | P < 0. [symbol] |
| | | (800) | Positive | | | | | 01 |
| | | | (250) | | | | | |
| Pfs28-CPF | Quil A | Negative | Negative | 20 | 75 | 55 | 3.8 | P < 0. [symbol] |
| | | | | | | | | 1 |
| Pfs28-CPF | Alhydrogel | Negative | Negative | 20 | 104 | 75 | 5.2 | P > 0. [symbol] |
| | | | | | | | | 5 |
| Pfs28-CPF | PBS | Negative | ND | 20 | 151 | 75 | 7.6 | P > 0. [symbol] |
| | | | | | | | | 5 |
| Preimmune | | | | 20 | 616 | 95 | 30.8 | ND |
| Sera GRP 1 | | | | | | | | |

Dose was 10 ug for each vaccine candidate
ND = not determined; AMO = arithmetic mean oocysts/feeder [symbol]
indicates data missing or illegible when filed

Analysis of the serum samples generated using the Pfs25-CPF demonstrated that high titers (1:6400) of parasite binding antibodies were detected in the adjuvanted groups with moderate titers (1:800) elicited by the vaccine alone. When analyzed on live parasites, all three Pfs25-CPF compositions, QUIL A™, ALHYDROGEL™, and PBS alone showed an antibody titer of 1:250. Similarly, when evaluated in the SMFA assay, all three Pfs25-CPF formulations elicited serum that completely blocked parasite transmission in the mosquitoes. These data confirm the potential of this approach in generating an efficacious vaccine against an infectious agent without the need for an adjuvant.

### EXAMPLE 5

### Preparation of Adenovirus Fusion Protein Constructs for Transfection of Mammalian Cells

For expression of the virus free particles (or fusion protein particles) composed of AIMV-CP-fusion proteins in a mammalian system, PCR techniques were used to fuse the Gaussia luciferase signal peptide to HA3A-CPF and HA3C04-CPF (globular domain (3) of HA from Anhui (SEQ ID NO:6), and California 04 (SEQ ID NO:7), respectively). The forward primers (SEQ ID NO:20 and 21) contained the TOPO integration sequence (CACC) and the nucleic acid sequence encoding the luciferase signal peptide and the start of the target protein. The reverse primer was specific to the AIMV-CP 3′ terminus (SEQ ID NO: 22). The resulting amplicons were cloned into the pENTR-D-TOPO vector according to manufacturer's instructions (Invitrogen Corporation). Sequences were confirmed by automated sequencing. Following sequence verification, the target genes were recombined into the pAd/CMV/V5-DEST vector using the Gateway technology from Invitrogen Corporation according to the manufacturer's instructions.

### EXAMPLE 6

### Preparation of Adenovirus Containing a Fusion Protein Gene

Purified pAd-HA3A-CPF or pAd-HA3C04-CPF plasmid was digested with Pac1 to expose the viral inverted terminal repeats (ITRs). The digested pAd-HA3A-CPF or pAd-HA3C04-CPF was transfected into the 293 cell line, which contains an integrated copy of E1 from adenovirus, using a cationic lipid-based transfection reagent. When the cytopathic effects of the virus were observed in transfected cells (10-14 days after transfection), adenovirus-containing cells and medium were harvested. The harvested cells and culture medium containing adenovirus were subjected to two rounds of freezing and thawing (at −80° C. for 30 min and then at 37° C. for 15 min) to lyse cells and allow the release of intracellular viral particles. After the second freeze/thaw cycle, the cell lysate was centrifuged and the supernatant containing viral particles was harvested and stored at −80° C.

### EXAMPLE 7

### Method for Transduction of the Adenoviral Construct with HA3A-CPF or HA3C04-CPF Sequences and Production of the Virus Free Particles

This is a prophetic example. The harvested adenovirus from Example 6 containing the HA3A-CPF or HA3C04-CPF gene will be amplified by infecting 293 cells, then again harvested and stored as described above to make an adenoviral stock. The adenoviral stock will be titrated using a plaque assay.

Since the pAd-HA3A-CPF and pAd-HA3C04-CPF adenoviral constructs are replication-incompetent and do not integrate into the host genome, no progeny virus will be produced after their transduction into mammalian cells. Host cells will be Madin-Darby canine kidney (MDCK) cells. MDCK cells will be adapted to grow in serum-free medium to avoid the possibility of animal by-product contamination in the final preparation. MDCK cells will be transduced with adenovirus-containing HA3A-CPF or HA3C04-CPF sequences to determine a suitable multiplicity of infection. Two or three days after transduction, the expression of HA3A-CPF or HA3C04-CPF will be analyzed by fluorescence microscopy using anti-HA and anti-AIMV antibodies followed by FITC-conjugated anti-mouse IgG. Alternatively, cell culture supernatant will be collected and concentrated for immunoblotting and detection of fusion proteins.

To obtain a scalable mammalian cell production platform for HA3A-CPF or HA3C04-CPF, MDCK cells will be cultured in suspension using a microcarrier system based on cross-linked dextran, e.g., CYTODEX™ 1 from GE Healthcare. After transduction of the adenovirus construct, MDCK cells will be incubated with the microcarrier and cultured in spinner flasks. After two to three days, samples of cell supernatants will be collected on a daily basis and expression levels will be analyzed by immunoblotting using a target specific anti-HA monoclonal antibody.

When the presence of fusion proteins is confirmed, the MDCK culture medium will be collected and centrifuged. Fusion proteins will be purified from the supernatant using the procedures described in Example 3, beginning with PEG precipitation.

### EXAMPLE 8

### Immunization of Mice with Virus Like Particles Containing Globular Domain of H5 HA (A/Indonesia/5/2005) AMV CP Fusion

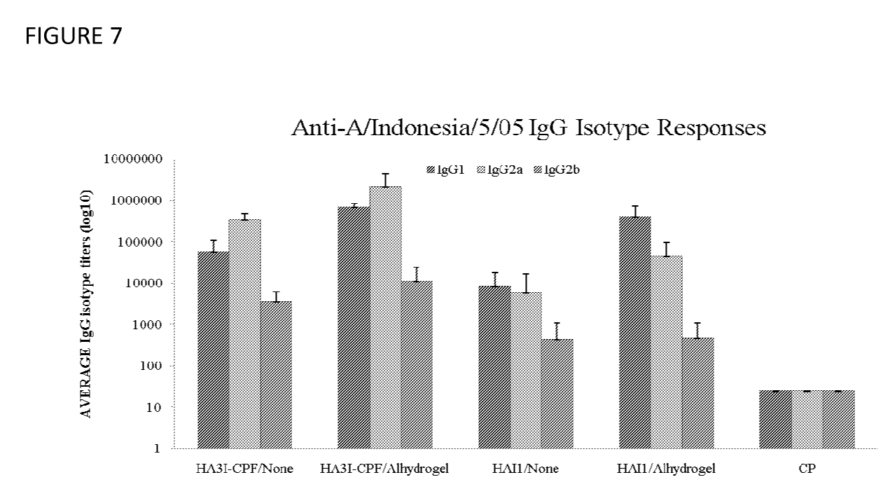

Groups of six, 6-8 weeks old Balb/c mice were immunized subcutaneously on study days 0 and 21 with 5 μg of virus like particles (VLPs) containing globular domain of H5 HA (A/Indonesia/5/2005) AMV CP fusion (HA3I-CPF). The full length soluble HA from the Indonesia strain of influenza virus (HAI1) was also injected with or without ALHYDROGEL™ as comparators. CP only was included as a negative control. The vaccine was prepared just before using. Aliquots of purified virus like particles (or fusion protein particles) were thawed in buffer and injected with or without an adjuvant, ALHYDROGEL™. Serum samples were collected on study day 35 and analyzed for antigen-specific IgG isotope responses by ELISA. In the assay, plates were coated with inactivated A/Indonesia/5/2005 reassortant virus obtained from the CDC at a 1:128 dilution. Serum samples were plated in 4-fold serial dilutions down the plate. End point titers of IgG1, IgG2a and IgG2b were determined as the dilution at which the corresponding OD values are greater than three times the negative control value. Results demonstrated that virus like particles containing HA3I-CPF induced stronger IgG2a response in the presence or absence of ALHYDROGEL™ than the soluble subunit alone (HAI1) in the presence or absence of ALHYDROGEL™ (FIG. 7) suggesting Th1 bias of immune response to VLPs. As mice infected with influenza have been shown to generate Th1 immune responses, plant-produced influenza HA globular domain AMV-based VLPs are better vaccine candidates than the soluble subunit.

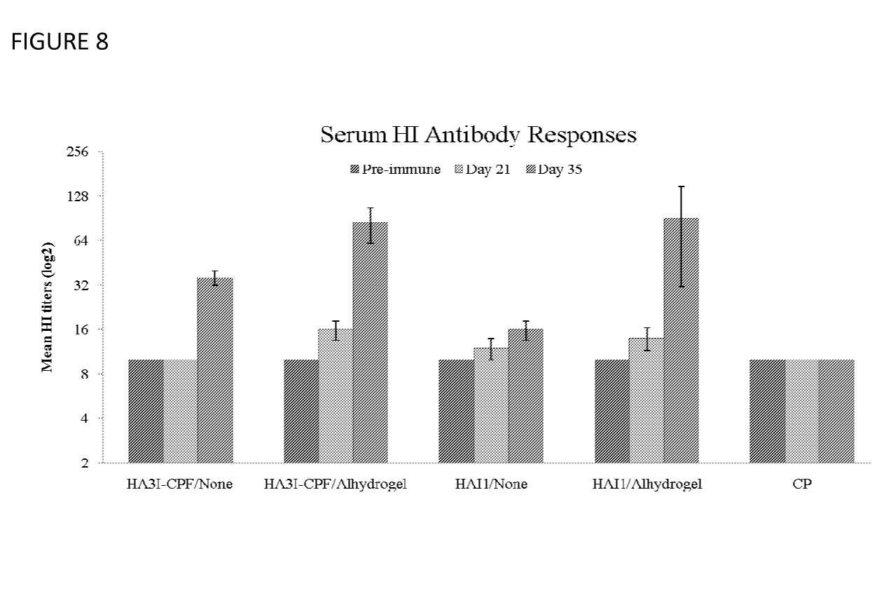

To test the functionality of the antibodies elicited by the HA3I-CPF vaccine, hemagglutination inhibition (HI) assays were performed as described in Yoko et al. (Vaccine 27: 3467-3470, 2009). Results showed that 80% of the mice 35 days after being immunized with HA3I-CPF in the absence of ALHYDROGEL™ had an HI titer of >1:40, which is significantly higher than the mice immunized with HAI1 (p=0.003) (FIG. 8), indicating that HA3I-CPF is a better influenza vaccine candidate than the soluble subunit for delivery without an adjuvant.

### EXAMPLE 9

### Effects of Pre-Existing Immunity Against CP on Serum Antibody Responses in Mice

Groups of six, 6-8 weeks old Balb/c mice were immunized subcutaneously (SC) or intramuscularly (IM) with PBS or 10 μg of virus like particles (VLPs) containing Pfs25-CPF on study days 0 and 28 with or without an adjuvant, ALHYDROGEL™ (Alum), and subsequently with PBS or 1 μg of VLPs containing globular domain of H5 HA (A/Anhui/1/2005) AMV CP fusion (HA3I-CPF) via the same route of administration on study days 140 and 168 with or without ALHYDROGEL™ (Alum).

The vaccine was prepared just before using. Aliquots of purified virus like particles (or fusion protein particles) were thawed in buffer and injected with or without an adjuvant, ALHYDROGEL™. Serum samples were collected before the study (Pre-bleed) and on study days 28, 56, 84, 112, 140, 168 and 216, and analyzed for antigen-specific IgG responses by ELISA. In the assay, plates were coated with 2 μg/ml AIMV, 1 μg/ml of recombinant Pfs25 produced in Pichia pastoris, or inactivated A/Anhui/1/2005 reassortant virus obtained from the CDC at a 1:128 dilution. Serum samples were plated in 4-fold dilutions down the plate. End point titers of IgG were determined as the dilution at which the corresponding OD values are greater than four times the blank value.

Two immunizations with Pfs25-CPF, either with or without an adjuvant, elicited high IgG antibody titers (>10,000) against AIMV (CP) and those titers were maintained throughout the course of the study. After immunizations with HA3A-CPF, either with or without an adjuvant, the IgG titers against AIMV (CP) in Pfs25-CPF pre-immunized group slightly increased while the IgG titers in PBS pre-immunized groups reached the equivalent levels to those in the Pfs25-CPF pre-immunized groups.

Two immunizations with Pfs25-CPF, either with or without an adjuvant, elicited high anti-Pfs25 IgG antibody titers (>10,000), which were maintained and did not significantly change after immunizations with Pfs25-CPF.

Two immunizations with Pfs25-CPF, by either the SC or IM route, elicited significant anti-A/Anhui/1/2005 IgG responses in sera from all groups except the groups pre-immunized with PBS in which the titers were one or two log lower than those in other groups (>10,000).

To test the functionality of the antibodies elicited by the HA3A-CPF vaccine, hemagglutination inhibition (HI) assays were performed as described in Yoko et al. (Vaccine 27: 3467-3470, 2009). Results showed that two immunizations with HA3A-CPF induced serum HI antibody responses in all groups except for the negative control (PBS/PBS) group (FIGS. 9A and 9B). Without an adjuvant, significant differences in HI titers between groups pre-immunized with Pfs25-CPF and PBS, either via SC (p=0.01) or IM (p=0.08) routes, were observed. With an adjuvant, there were no significant differences observed between groups pre-immunized with Pfs25-CPF and PBS via SC (p=0.5) or IM (p=0.6). Based on these results, without ALHYDROGEL™ as an adjuvant, pre-immunization with VLPs containing a fusion protein of a plant viral coat protein and a first target protein did not interfere with serum HI antibody responses to a second target protein in mice immunized with VLPs containing a fusion protein of the same plant viral coat protein and the second target protein, via SC or IM routes.

Although the invention is illustrated and described herein with reference to specific embodiments, the invention is not intended to be limited to the details shown. Rather, various modifications may be made in the details within the scope and range of equivalents of the claims and without departing from the invention.

1. A virus like particle comprising a fusion protein, wherein the fusion protein comprises a plant viral coat protein and a target protein, and wherein the plant viral coat protein is derived from a coat protein of a plant virus selected from the group consisting of alfalfa mosaic virus, Johnsongrass mosaic virus, and potato virus Y.

2. (canceled)

3. The virus like particle of claim 1, wherein the target protein is derived from a cell surface polypeptide of an intracelluar pathogen.

4. The virus like particle of claim 1, wherein the target protein is derived from a cell surface polypeptide of Plasmodium falciparum

5. The virus like particle of claim 1, wherein the target protein is derived from a polypeptide of hemagglutinin of an influenza virus.

6. The virus like particle of claim 5, wherein the influenza virus is an influenza A virus selected from the group consisting of H1N1, H3N2, H5N1, and H7N7.

7. The virus like particle of claim 5, wherein the influenza virus is an influenza B virus.

8. The virus like particle of claim 1, wherein the target protein comprises an amino acid sequence selected from the group consisting of SEQ ID NOs: 4-11 and 49, and antigenic fragments thereof.

9. The virus like particle of claim 1, wherein the target protein is fused to the N-terminus of the plant viral coat protein.

10. The virus like particle of claim 1, wherein the virus like particle is produced in a host cell selected from the group consisting of bacterial, fungal, plant, insect, amphibian, and mammalian cells.

11. An immunogenic composition comprising a virus like particle, wherein the virus like particle comprises a fusion protein, wherein the fusion protein comprises a plant viral coat protein and a target protein, and wherein the plant viral coat protein is derived from a coat protein of a plant virus selected from the group consisting of alfalfa mosaic virus, Johnsongrass mosaic virus, and potato virus Y.

12. The immunogenic composition of claim 11, further comprising an adjuvant.

13. The immunogenic composition of claim 12, wherein the adjuvant is selected from the group consisting of aluminum salts, oil and water emulsions, and saponin-based adjuvants.

14. (canceled)

15. The immunogenic composition of claim 11, wherein the target protein is derived from an intracellular pathogen.

16. The immunogenic composition of claim 11, wherein the target protein is derived from a cell surface polypeptide of Plasmodium falciparum or a polypeptide of hemagglutinin of an influenza virus.

17. The immunogenic composition of claim 11, wherein the plant viral coat protein is derived from a coat protein of an alfalfa mosaic virus and the target protein comprises an amino acid sequence selected from the group consisting of SEQ ID NOs: 4-11 and 49, and antigenic fragments thereof.

18. A method for inducing a protective immune response against a first intracellular pathogen in a subject, comprising administering to the subject an immunologically effective amount of a first composition comprising a first virus like particle, wherein the first virus like particle comprises a first fusion protein, wherein the first fusion protein comprises a plant viral coat protein and a first target protein derived from the first intracellular pathogen, and wherein the plant viral coat protein is derived from a coat protein of a plant virus selected from the group consisting of alfalfa mosaic virus, Johnsongrass mosaic virus, and potato virus Y.

19. The method of claim 18, wherein the plant viral coat protein is derived from a coat protein of an alfalfa mosaic virus and the first target protein comprises an amino acid sequence selected from the group consisting of SEQ ID NOs: 4-11 and 49, and antigenic fragments thereof.

20. (canceled)

21. The method of claim 18, further comprising administering to the subject an immunologically effective amount of a second composition comprising a second virus like particle prior to the administering the first composition, wherein the second virus like particle comprises a second fusion protein, and wherein the second fusion protein comprises the plant viral coat protein and a second target protein.

22. The method of claim 21, wherein the second target protein is derived from a second intracellular pathogen that is not the first intracellular pathogen.

23. The method of claim 21, wherein the first target intracellular pathogen is an influenza virus, the plant viral coat protein is derived from a coat protein of an alfalfa mosaic virus, the first target protein comprises an amino acid sequence of SEQ ID NO: 6, and the second target protein comprises an amino acid sequence of SEQ ID NO: 4.

24-29. (canceled)
